SEQUENCE LISTING (70 sequences)

SEQ ID NO: 1 - protein, 10 aa, Homo sapiens
GGSVSRGSHY

SEQ ID NO: 2 - protein, 7 aa, Homo sapiens
IHTDGST

SEQ ID NO: 3 - protein, 10 aa, Homo sapiens
AREPGGALNF

SEQ ID NO: 4 - protein, 8 aa, Homo sapiens
GYTFTGYY

SEQ ID NO: 5 - protein, 8 aa, Homo sapiens
INPNSGGT

SEQ ID NO: 6 - protein, 19 aa, Homo sapiens
ASGKTYYDFWSGGRRGMDV

SEQ ID NO: 7 - protein, 8 aa, Homo sapiens
GGTFSSYA

SEQ ID NO: 8 - protein, 8 aa, Homo sapiens
IIPIFGTA

SEQ ID NO: 9 - protein, 15 aa, Homo sapiens
ARSQVPSSYYYGMDV

SEQ ID NO: 10 - protein, 8 aa, Homo sapiens
GFTFSSYA

SEQ ID NO: 11 - protein, 8 aa, Homo sapiens
ISYDGSNK

SEQ ID NO: 12 - protein, 12 aa, Homo sapiens
ARGREYYYYMDV

SEQ ID NO: 13 - protein, 8 aa, Homo sapiens
GYTFTSYY

SEQ ID NO: 14 - protein, 8 aa, Homo sapiens
INPSGGST

SEQ ID NO: 15 - protein, 13 aa, Homo sapiens
ARGFGYGGKALDY

SEQ ID NO: 16 - protein, 8 aa, Homo sapiens
SSNIGSNT

SEQ ID NO: 17 - protein, 11 aa, Homo sapiens
ASWDDSLNGVV

SEQ ID NO: 18 - protein, 6 aa, Homo sapiens
QSVSRY

SEQ ID NO: 19 - protein, 9 aa, Homo sapiens
QQTYSTPWT

SEQ ID NO: 20 - protein, 9 aa, Homo sapiens
NSNIGAGYH

SEQ ID NO: 21 - protein, 11 aa, Homo sapiens
QSYDRSRGGWF

SEQ ID NO: 22 - protein, 6 aa, Homo sapiens
NIARKS

SEQ ID NO: 23 - protein, 9 aa, Homo sapiens
QVWDNSDYV

SEQ ID NO: 24 - protein, 6 aa, Homo sapiens
QNINSR

SEQ ID NO: 25 - protein, 9 aa, Homo sapiens
QQFDKYSIT

SEQ ID NO: 26 - DNA, 30 bp, Homo sapiens
  source 1..30: mol_type = other DNA
ggtggctccgtcagcagaggtagtcactac

SEQ ID NO: 27 - DNA, 21 bp, Homo sapiens
  source 1..21: mol_type = other DNA
atccacaccgatgggagcacc

SEQ ID NO: 28 - DNA, 30 bp, Homo sapiens
  source 1..30: mol_type = other DNA
gcgagagagcccgggggcgccctgaatttc

SEQ ID NO: 29 - DNA, 24 bp, Homo sapiens
  source 1..24: mol_type = other DNA
ggatacaccttcaccggctactat

SEQ ID NO: 30 - DNA, 24 bp, Homo sapiens
  source 1..24: mol_type = other DNA
atcaaccctaacagtggtggcaca

SEQ ID NO: 31 - DNA, 57 bp, Homo sapiens
  source 1..57: mol_type = other DNA
gcgagtgggaaaacgtattacgatttttggagtggtgggagacgcggtatggacgtc

SEQ ID NO: 32 - DNA, 24 bp, Homo sapiens
  source 1..24: mol_type = other DNA
ggaggcaccttcagcagctatgct

SEQ ID NO: 33 - DNA, 24 bp, Homo sapiens
  source 1..24: mol_type = other DNA
atcatccctatctttggtacagca

SEQ ID NO: 34 - DNA, 45 bp, Homo sapiens
  source 1..45: mol_type = other DNA
gcgagaagccaggtaccatcctcctactactacggtatggacgtc

SEQ ID NO: 35 - DNA, 24 bp, Homo sapiens
  source 1..24: mol_type = other DNA
ggattcaccttcagtagctatgct

SEQ ID NO: 36 - DNA, 24 bp, Homo sapiens
  source 1..24: mol_type = other DNA
atatcatatgatggaagcaataaa

SEQ ID NO: 37 - DNA, 36 bp, Homo sapiens
  source 1..36: mol_type = other DNA
gcgagaggtcgtgaatactactactacatggacgtc

SEQ ID NO: 38 - DNA, 24 bp, Homo sapiens
  source 1..24: mol_type = other DNA
ggatacaccttcaccagctactat

SEQ ID NO: 39 - DNA, 24 bp, Homo sapiens
  source 1..24: mol_type = other DNA
atcaaccctagtggtggtagcaca

SEQ ID NO: 40 - DNA, 39 bp, Homo sapiens
  source 1..39: mol_type = other DNA
gcgagaggattcggctacggtggtaaggcccttgactac

SEQ ID NO: 41 - DNA, 24 bp, Homo sapiens
  source 1..24: mol_type = other DNA
agctccaacatcggaagtaatact

SEQ ID NO: 42 - DNA, 33 bp, Homo sapiens
  source 1..33: mol_type = other DNA
gcatcatgggatgacagcctgaatggtgtggta

SEQ ID NO: 43 - DNA, 18 bp, Homo sapiens
  source 1..18: mol_type = other DNA
cagagcgttagcaggtat

SEQ ID NO: 44 - DNA, 27 bp, Homo sapiens
  source 1..27: mol_type = other DNA
caacagacttacagtaccccgtggacg

SEQ ID NO: 45 - DNA, 27 bp, Homo sapiens
  source 1..27: mol_type = other DNA
aactccaacatcggggcaggttatcat

SEQ ID NO: 46 - DNA, 33 bp, Homo sapiens
  source 1..33: mol_type = other DNA
cagtcctatgacagaagccggggtggttggttt

SEQ ID NO: 47 - DNA, 18 bp, Homo sapiens
  source 1..18: mol_type = other DNA
aacattgcaagaaaaagt

SEQ ID NO: 48 - DNA, 27 bp, Homo sapiens
  source 1..27: mol_type = other DNA
caggtgtgggataatagtgattatgtc

SEQ ID NO: 49 - DNA, 18 bp, Homo sapiens
  source 1..18: mol_type = other DNA
cagaatattaatagtagg

SEQ ID NO: 50 - DNA, 27 bp, Homo sapiens
  source 1..27: mol_type = other DNA
caacagtttgataaatattcgatcact

SEQ ID NO: 51 - DNA, 354 bp, Homo sapiens
  source 1..354: mol_type = other DNA
caggtacagctgcagcagttgggcccaggactggtgaagccttcggagaccctgtccctcacctgcaatgtctctggtggctccgtcagcagaggtagtcactactggacctggatccggcagcccccaggaaagggactggagtggattgggtatatccacaccgatgggagcaccaacttcaatccctccctcaagagtcgagtcaccatgtcactagacaggtccaggaaccagttctccctgacgctgagctctgtgaccgctacggacacggccgtttattattgtgcgagagagcccgggggcgccctgaatttctggggccagggaaccctggtcaccgtctcctca

SEQ ID NO: 52 - DNA, 330 bp, Homo sapiens
  source 1..330: mol_type = other DNA
aattttatgctgactcagccaccctcaacgtctgggacccccgggcagagggtcaccatctcttgttctggaagcagctccaacatcggaagtaatactgtaaactggtaccagcagctcccaggaacggcccccaaactcctcatctatagtaataatcagcggccctcaggggtccctgaccgattctctggctccaagtctggcacctcagcctccctggccatcagtgggctccagtctgaggatgaggctgattattattgtgcatcatgggatgacagcctgaatggtgtggtattcggcggagggaccacggtgaccgtcctg

SEQ ID NO: 53 - DNA, 378 bp, Homo sapiens
  source 1..378: mol_type = other DNA
caggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggacggatcaaccctaacagtggtggcacaaactatgcacagaagtttcagggcagggtcaccatgaccagggacacgtccatcagcacagcctacatggagctgagcaggctgagatctgacgacacggccgtgtattactgtgcgagtgggaaaacgtattacgatttttggagtggtgggagacgcggtatggacgtctggggccaagggacaatggtcaccgtctcttca

SEQ ID NO: 54 - DNA, 321 bp, Homo sapiens
  source 1..321: mol_type = other DNA
gacatccagttgacccagtctccatcctccctgtctgcatctgtaggagacagagtcaccatcacttgccgggcaggtcagagcgttagcaggtatttaaattggtttcagcagaaaccagggaaagcccctaagctcctcatctatacttcatccaatttgcaaagtggggtcccatcaaggttcagtgccagtggatctgggacagatttcactctcaccatcagcagtctgcaacctgaggattttgctacctactactgtcaacagacttacagtaccccgtggacgttcggccaagggaccaagctggagatcaaa

SEQ ID NO: 55 - DNA, 366 bp, Homo sapiens
  source 1..366: mol_type = other DNA
caggtgcagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgcggacgaatccacgagcacagcctacatggagctgagcagcctgagatctgaggacacggccgtgtattactgtgcgagaagccaggtaccatcctcctactactacggtatggacgtctggggccaagggacaatggtcaccgtctcctca

SEQ ID NO: 56 - DNA, 333 bp, Homo sapiens
  source 1..333: mol_type = other DNA
cagtctgtgctgactcagccgccctcagtgtctggggccccagggcagagggtcaccatctcctgcactgggggcaactccaacatcggggcaggttatcatgtacattggtaccagcaacttccaggagcagcccccaaactcctcatctatggtgacactaatcggccctcaggggtccctgaccgattctctggctctcagtctggcacctcagcctccctggccatcactgggctccaggctgacgatgaggctgattattactgccagtcctatgacagaagccggggtggttggtttttcggcggagggacccagctgaccgtccta

SEQ ID NO: 57 - DNA, 357 bp, Homo sapiens
  source 1..357: mol_type = other DNA
caggtgaagctggtggagtggtcgctgagcgtggtccagcctgggaggtccctgagactctcctgcgcagcctctggattcaccttcagtagctatgctatgcactgggtccgccaggctccaggcaaggggctggagtgggtggcagttatatcatatgatggaagcaataaatactacgcagactccgtgaagggccgattcaccatctccagagacaattccaagaacacgctgtatctgcaaatgaacagcctgagagctgaggacacggctgtgtattactgtgcgagaggtcgtgaatactactactacatggacgtctggggcaaagggaccacggtcagcgtctcctca

SEQ ID NO: 58 - DNA, 318 bp, Homo sapiens
  source 1..318: mol_type = other DNA
cagtctgtgctgactcagccaccctcagtgtcagtggccccaggaaagacggccaggattacatgtgggggatacaacattgcaagaaaaagtgtgcactggtaccagcagaagccaggccaggcccctgtgttggtcatgtataatgataacgaccggccctcagggatccctgagcgattctctggctccaactctgggaacacggccaccctgaccatcagcagggtcgaagccggggatgaggccgactattactgtcaggtgtgggataatagtgattatgtcttcggaactgggaccaaggtcaccgtccta

SEQ ID NO: 59 - DNA, 360 bp, Homo sapiens
  source 1..360: mol_type = other DNA
caggtgcagttgatgcagtctggggctgaggtgaagaagcctggggcctcagtgaaggtttcctgcaaggcatctggatacaccttcaccagctactatatgcactgggtgcgacaggcccctggacaagggcttgagtggatgggaataatcaaccctagtggtggtagcacaagctacgcacagaagttccagggcagagtcaccatgaccagggacacgtccacgagcacagtctacatggagctgagcagcctgagatctgaggacacggccgtgtattactgtgcgagaggattcggctacggtggtaaggcccttgactactggggccagggaaccacggtcaccgtctcttca

SEQ ID NO: 60 - DNA, 321 bp, Homo sapiens
  source 1..321: mol_type = other DNA
gacatccagatgacccagtctcctcccaccctgtctgcatctgtaggggacagagtcaccatcacttgccgggccagtcagaatattaatagtaggttggcctggtatcagcagaaaccagggagagcccctaaactcctgatctataaggcgtctactttagagagtggggtcccatcgaggttcagcggcagtggatctgggacagaattcactctcaccatcagcagcctgcagccagatgactttgcaacttattactgccaacagtttgataaatattcgatcactttcggcggagggaccaagatggagatcaaa

SEQ ID NO: 61 - protein, 118 aa, Homo sapiens
QVQLQQLGPGLVKPSETLSLTCNVSGGSVSRGSHYWTWIRQPPGKGLEWIGYIHTDGSTNFNPSLKSRVTMSLDRSRNQFSLTLSSVTATDTAVYYCAREPGGALNFWGQGTLVTVSS

SEQ ID NO: 62 - protein, 110 aa, Homo sapiens
DFMLTQPPSTSGTPGQRVTISCSGSSSNIGSNTVNWYQQLPGTAPKLLIYSNNQRPSGVPDRFSGSKSGTSASLAISGLQSEDEADYYCASWDDSLNGVVFGGGTTVTVL

SEQ ID NO: 63 - protein, 126 aa, Homo sapiens
QVQLVQSGAEVKKPGASVKVSCKASGYTFTGYYMHWVRQAPGQGLEWMGRINPNSGGTNYAQKFQGRVTMTRDTSISTAYMELSRLRSDDTAVYYCASGKTYYDFWSGGRRGMDVWGQGTMVTVSS

SEQ ID NO: 64 - protein, 107 aa, Homo sapiens
DIQLTQSPSSLSASVGDRVTITCRAGQSVSRYLNWFQQKPGKAPKLLIYTSSNLQSGVPSRFSASGSGTDFTLTISSLQPEDFATYYCQQTYSTPWTFGQGTKLEIK

SEQ ID NO: 65 - protein, 122 aa, Homo sapiens
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARSQVPSSYYYGMDVWGQGTMVTVSS

SEQ ID NO: 66 - protein, 111 aa, Homo sapiens
QSVLTQPPSVSGAPGQRVTISCTGGNSNIGAGYHVHWYQQLPGAAPKLLIYGDTNRPSGVPDRFSGSQSGTSASLAITGLQADDEADYYCQSYDRSRGGWFFGGGTQLTVL

SEQ ID NO: 67 - protein, 119 aa, Homo sapiens
QVKLVEWSLSVVQPGRSLRLSCAASGFTFSSYAMHWVRQAPGKGLEWVAVISYDGSNKYYADSVKGRFTISRDNSKNTLYLQMNSLRAEDTAVYYCARGREYYYYMDVWGKGTTVSVSS

SEQ ID NO: 68 - protein, 106 aa, Homo sapiens
QSVLTQPPSVSVAPGKTARITCGGYNIARKSVHWYQQKPGQAPVLVMYNDNDRPSGIPERFSGSNSGNTATLTISRVEAGDEADYYCQVWDNSDYVFGTGTKVTVL

SEQ ID NO: 69 - protein, 120 aa, Homo sapiens
QVQLMQSGAEVKKPGASVKVSCKASGYTFTSYYMHWVRQAPGQGLEWMGIINPSGGSTSYAQKFQGRVTMTRDTSTSTVYMELSSLRSEDTAVYYCARGFGYGGKALDYWGQGTTVTVSS

SEQ ID NO: 70 - protein, 107 aa, Homo sapiens
DIQMTQSPPTLSASVGDRVTITCRASQNINSRLAWYQQKPGRAPKLLIYKASTLESGVPSRFSGSGSGTEFTLTISSLQPDDFATYYCQQFDKYSITFGGGTKMEIK

(19) United States
(12) Patent Application Publication
(10) Pub. No.: US 2025/0042991 A1
(43) Pub. Date: Feb. 6, 2025

(54) LEPTIN ANTIBODIES AND METHOD OF REDUCING LEPTIN

(71) Applicant: BOARD OF REGENTS, THE UNIVERSITY OF TEXAS SYSTEM, Austin, TX (US)

(72) Inventors: Zhiqiang AN, Houston, TX (US); Ningyang ZHANG, Houston, TX (US); Philipp E. SCHERER, Dallas, TX (US); Shangang ZHAO, Dallas, TX (US)

(73) Assignee: BOARD OF REGENTS, THE UNIVERSITY OF TEXAS SYSTEM, Austin, TX (US)

(21) Appl. No.: 18/765,613

(22) Filed: Jul. 8, 2024

Related U.S. Application Data
(62) Division of application No. 18/365,969, filed on Aug. 4, 2023, now Pat. No. 12,037,390.
Application No. 18/365,969 is a division of application No. 17/124,481, filed on Dec. 16, 2020, now Pat. No. 11,760,799.
Application No. 17/124,481 is a continuation of application No. PCT/US19/37236, filed on Jun. 14, 2019.
(60) Provisional application No. 62/685,997, filed on Jun. 16, 2018.

(51) Int. Cl. C07K 16/26; A61K 39/00; A61P 3/04

(52) U.S. Cl. CPC C07K 16/26; A61P 3/04; A61K 2039/505; C07K 2317/24; C07K 2317/76; C07K 2317/92

(57) ABSTRACT

Antibody antigen binding domains which specifically binds human leptin comprises V_{H} or V_{L} CDR1, CDR2 and CDR3 sequences of an hLept antibody. The antibody antigen binding domains and antibodies thereof are useful to treat obesity and diabetes.

This application is a continuation of U.S. patent application Ser. No. 18/365,969, filed Aug. 4, 2023, which is a divisional of U.S. patent application Ser. No. 17/124,481, now issued as U.S. Pat. No. 11,760,799, filed Dec. 16, 2020, which is a continuation of International Application No. PCT/US19/37236, filed Jun. 14, 2019, which claims the priority benefit of U.S. Provisional Patent Application No. 62/685,997, filed Jun. 16, 2018, the entirety of each of which are incorporated herein by reference.

### REFERENCE TO A SEQUENCE LISTING

This application contains a Sequence Listing XML, which has been submitted electronically and is hereby incorporated by reference in its entirety. Said XML Sequence Listing, created on Jul. 5, 2024, is named UTSHP0405USC1.xml and is 66,065 bytes in size.

### INTRODUCTION

Leptin is a hormone produced by adipocytes and is elevated in obesity. The congenital lack of leptin results in obesity and the metabolism field widely accepts the concept. Upon cloning of the leptin gene, the original hope was that leptin would act as a break for further food intake and a trigger to increase energy expenditure. The hope was that the injection of recombinant leptin would act as an effective weight loss mechanism. However, these hopes were quickly disappointed, since obese individuals have high leptin levels, but the individual is leptin resistant. Not even the injection of very high leptin levels can overcome this resistance.

We generated a battery of monoclonal antibodies against human and mouse leptin. These antibodies showed strong binding affinities to human leptin and significant neutralizing activity in vivo. More importantly, treatment of high-fat diet (HFD)-fed mice with neutralizing antibodies reduces body-weight gain and confirmed findings in mice with genetic knock-down of leptin. Our antibodies provide effective for treatment of obesity with high level of leptin but resistant to the conventional leptin treatment.

### SUMMARY OF THE INVENTION

In an aspect the invention provides an antibody antigen binding domain which specifically binds human leptin, and comprises V_{H} or V_{L} CDR1, CDR2 and CDR3 sequences of an hLept antibody:

| | CDR1 | CDR2 | CDR3 |
|---|---|---|---|
| V_{H} | | | |
| hLept-1V_{H} | GGSVSRGSHY | IHTDGST | AREPGGALNF |
| | (SEQ ID NO: 1) | (SEQ ID NO: 2) | (SEQ ID NO: 3) |
| hLept-2V_{H} | GYTFTGYY | INPNSGGT | ASGKTYYDFWSGGRRGMDV |
| | (SEQ ID NO: 4) | (SEQ ID NO: 5) | (SEQ ID NO: 6) |
| hLept-3V_{H} | GGTFSSYA | IIPIFGTA | ARSQVPSSYYYGMDV |
| | (SEQ ID NO: 7) | (SEQ ID NO: 8) | (SEQ ID NO: 9) |
| hLept-5V_{H} | GFTFSSYA | ISYDGSNK | ARGREYYYYMDV |
| | (SEQ ID NO: 10) | (SEQ ID NO: 11) | (SEQ ID NO: 12) |
| hLept-6V_{H} | GYTFTSYY | INPSGGST | ARGFGYGGKALDY |
| | (SEQ ID NO: 13) | (SEQ ID NO: 14) | (SEQ ID NO: 15) |
| V_{L} | | | |
| hLept-1V_{L} | SSNIGSNT | SNN | ASWDDSLNGVV |
| | (SEQ ID NO: 16) | | (SEQ ID NO: 17) |
| hLept-2V_{L} | QSVSRY | TSS | QQTYSTPWT |
| | (SEQ ID NO: 18) | | (SEQ ID NO: 19) |
| hLept-3V_{L} | NSNIGAGYH | GDT | QSYDRSRGGWF |
| | (SEQ ID NO: 20) | | (SEQ ID NO: 21) |
| hLept-5V_{L} | NIARKS | NDN | QVWDNSDYV |
| | (SEQ ID NO: 22) | | (SEQ ID NO: 23) |
| hLept-6V_{L} | QNINSR | KAS | QQFDKYSIT |
| | (SEQ ID NO: 24) | | (SEQ ID NO: 25) |

The V_{H} and V_{L} CDR1, CDR2 and CDR3 sequences of the hLept antibodies can be combined in alternative combinations which bind human leptin, i.e. the CDRs of hLept-1V_{H} can be paired with the CDRs of hLept-1-V_{L}, hLept-2V_{L}, hLept-3V_{L}, hLept-5V_{L or} hLept-6V_{L}.

In embodiments the antigen binding domain comprises:
V_{H} and V_{L} CDR1, CDR2 and CDR3 sequences of the hLept antibody;
V_{H} or V_{L} sequences of the hLept antibody; and/or
V_{H} and V_{L} sequences of the hLept antibody.

In embodiments the antigen binding domain is part of a monoclonal IgG antibody and/or a humanized antibody.

In other aspects the invention provides an expression vector encoding the antibody antigen binding domain or a cultured cell expressing the antibody antigen binding domain.

In another aspect the invention provides a method of using the antibody antigen binding domain to treat obesity or diabetes, comprising the step of administering the domain to a person in need thereof.

The invention includes all combinations of the recited particular embodiments.

### BRIEF DESCRIPTION OF THE DRAWINGS

FIG. 1A. Antibody therapy reduces body weight.

FIG. 1B. Antibody therapy reduces food intake.

FIG. 1C. Antibody therapy increases leptin transcription in brown and epididymal adipose tissue.

FIG. 1D. Antibody therapy improves glucose homeostasis.

FIG. 1E. Antibody therapy reduces high fat diet effects on brown adipose tissue.

### DESCRIPTION OF PARTICULAR EMBODIMENTS OF THE INVENTION

Unless the context indicates otherwise, the term “antibody” is used in the broadest sense and specifically covers antibodies (including full length monoclonal antibodies) and antibody fragments. An antibody molecule is usually monospecific, but may also be described as idiospecific, heterospecific, or polyspecific. Antibody molecules bind by means of specific binding sites to specific antigenic determinants or epitopes on antigens. “Antibody fragments” comprise a portion of a full length antibody, generally the antigen binding or variable region thereof. Examples of antibody fragments include Fab, Fab′, F(ab′).sub.2, and Fv fragments; diabodies; linear antibodies; single-chain antibody molecules; and multispecific antibodies formed from antibody fragments.

Natural and engineered antibody structures are well known in the art, e.g. Strohl et al., Therapeutic antibody engineering: Current and future advances driving the strongest growth area in the pharmaceutical industry, Woodhead Publishing Series in Biomedicine No. 11, October 2012; Holliger et al. Nature Biotechnol 23, 1126-1136 (2005); Chames et al. Br J Pharmacol. 2009 May; 157(2): 220-233.

Monoclonal antibodies (MAbs) may be obtained by methods known to those skilled in the art. See, for example Kohler et al (1975); U.S. Pat. No. 4,376,110; Ausubel et al (1987-1999); Harlow et al (1988); and Colligan et al (1993). The mAbs of the invention may be of any immunoglobulin class including IgG, IgM, IgE, IgA, and any subclass thereof. A hybridoma producing a mAb may be cultivated in vitro or in vivo. High titers of mAbs can be obtained in in vivo production where cells from the individual hybridomas are injected intraperitoneally into mice, such as pristine-primed Balb/c mice to produce ascites fluid containing high concentrations of the desired mAbs. MAbs of isotype IgM or IgG may be purified from such ascites fluids, or from culture supernatants, using column chromatography methods well known to those of skill in the art.

An “isolated polynucleotide” refers to a polynucleotide segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA, which is part of a hybrid gene encoding additional polypeptide sequence.

A “construct” means any recombinant polynucleotide molecule such as a plasmid, cosmid, virus, autonomously replicating polynucleotide molecule, phage, or linear or circular single-stranded or double-stranded DNA or RNA polynucleotide molecule, derived from any source, capable of genomic integration or autonomous replication, comprising a polynucleotide molecule where one or more polynucleotide molecule has been linked in a functionally operative manner, i.e. operably linked. A recombinant construct will typically comprise the polynucleotides of the invention operably linked to transcriptional initiation regulatory sequences that will direct the transcription of the polynucleotide in the intended host cell. Both heterologous and non-heterologous (i.e., endogenous) promoters can be employed to direct expression of the nucleic acids of the invention.

A “vector” refers any recombinant polynucleotide construct that may be used for the purpose of transformation, i.e. the introduction of heterologous DNA into a host cell. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”.

An “expression vector” as used herein refers to a nucleic acid molecule capable of replication and expressing a gene of interest when transformed, transfected or transduced into a host cell. The expression vectors comprise one or more phenotypic selectable markers and an origin of replication to ensure maintenance of the vector and to, if desired, provide amplification within the host. The expression vector further comprises a promoter to drive the expression of the polypeptide within the cells. Suitable expression vectors may be plasmids derived, for example, from pBR322 or various pUC plasmids, which are commercially available. Other expression vectors may be derived from bacteriophage, phagemid, or cosmid expression vectors.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein, including citations therein, are hereby incorporated by reference in their entirety for all purposes.

### EXAMPLES

### Panning and Selection of Anti-Leptin Monoclonal Antibodies Using In-House Prepared scFv Phage Display Human Antibody Library

Leptin protein (NM-000230, Entrez 3952: Sino Biologics) was used to panning the phage display scFv library (diversity of 1×10¹¹). Binders were selected by phage ELISA by coating LEPTIN protein on 96-well plates (max-sorb plates, Nunc) and were detected with an anti-M13 phage antibody conjugated with horseradish peroxidase (HRP) and TMB substrate (cell signaling). DNA sequences contained in phage-mid vector is isolated using a plasmid preparation kit and sequenced (Genewiz). Complete heavy chain variable region and light chain variable sequences were amplified and expressed into full IgG using an expression vector system in HEK293 cells.

Selected LEPTIN binding hits were expressed as human IgGs using a mammalian expression vector system in human embryonic kidney (HEK293) cells (Invitrogen). Antibodies were purified using a column with protein A resin by a fast protein liquid chromatography (FPLC) separation unit. Purified LEPTIN binding antibodies were characterized for their biological properties.

### Binding Affinity of Anti-Leptin Monoclonal Antibodies

Binding of LEPTIN by monoclonal antibodies was first screened by ELISA using supernatants collected from the B cell cultures. Human or mouse LEPTIN protein (Sino Biologicals) was coated on a 96-well high binding plate over night at 4° C. in PBS. B cell culture supernatants (5 μl medium and 95 μl of PBS) were added at for binding to LEPTIN antigen coated on the plate. Bound antibody was detected using a secondary antibody against rabbit IgG conjugated with HRP and TMB substrate.

Binding to human leptin in ELISA (1:20 diluted culture supernatants; signals: absorbance at 450 nM):

| hLept-1/ | hLept-2/ | hLept-3/ | hLept-5/ | hLept-6/ |
|---|---|---|---|---|
| 0.071 | 2.5739 | 0.1902 | 0.7385 | 0.663 |

ELISA titration was used to determine the binding affinity of a panel of monoclonal antibodies to LEPTIN antigen. Binding constants (K_{D} and/or EC 50) of a panel of monoclonal antibodies were estimated using the 4 parameter curve fitting with Prism GraphPad program. For Biacore analysis, all experiments were performed at 25° C. at a flow rate of 45 μl/min. An anti-human IgG Fc antibody (from ThermoFisher, at 50 μg/ml each in acetate buffer, pH 5.0) was immobilized onto a carboxymethyl dextran sensorchip (CM5) using amine coupling procedures based on instruction from the manufacturer. Purified rabbit/human chimeric antibody to be tested was diluted at a concentration of 5 μg/ml in 0.5% P20, HBS-EP buffer and injected on FC2 to reach 500 to 1000 RU. FC1 was used as the reference cell. Specific signals correspond to the difference of signals obtained on FC2 versus FC1. The analyte (recombinant human LEPTIN, apparent molecular weight 16 kDa on SDS-PAGE gel) was injected during 90 sec at series of concentration dilutions (100, 50, 25, 12.5, 6.25, and 3.13, 1.56 nM) in 0.5% P20, HBS-EP buffer. These concentrations were prepared from stock solution in 0.5% P20, HBS-EP. The dissociation phase of the analyte was monitored over a 30 minutes period. Running buffer was also injected under the same conditions as a double reference. After each running cycle, both flow cells were regenerated by injecting 20 to 45 μl of Glycine-HCl buffer pH 1.5. Binding K_{D} on LEPTIN was calculated by k_{off}/kₒₙ kinetic rate for each LEPTIN monoclonal antibodies (Table 3).

### Neutralizing Leptin Antibodies Confer Reduced Weight Gain

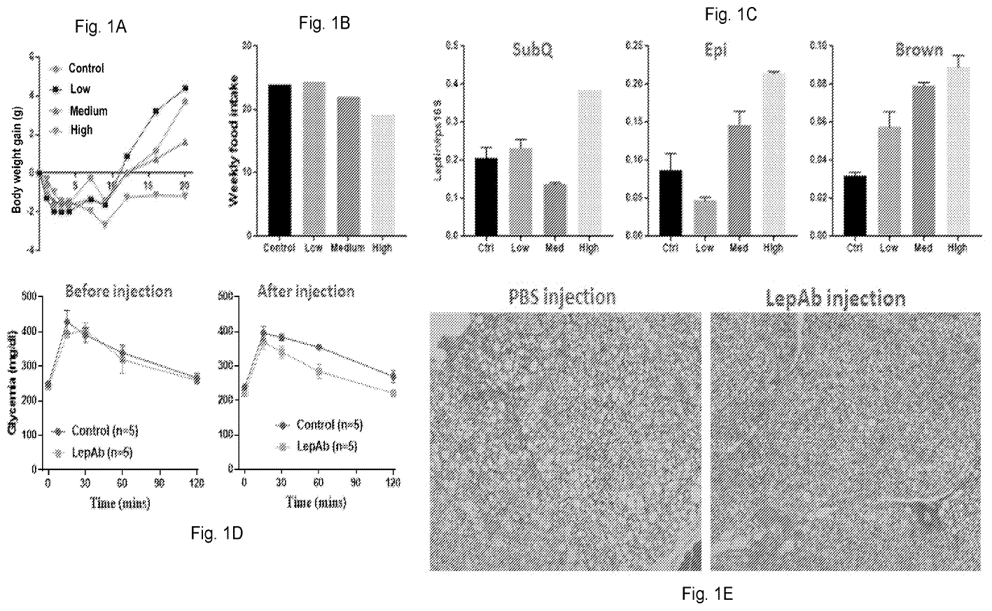

We used a cohort of mice that had previously been exposed to 10 weeks of high fat diet exposure. We treated these mice twice a week either with PBS, low (“0.5 microgram/g BW), medium (5 microgram/g BW) or high (50 microgram/g BW) leptin antibody injections for up to 20 days; hLept-1, hLept-2, hLetp-3, hLept-5 and hLept-6 antibody injections provide consistent dose dependent results. As seen in FIG. 1A, there is a dose-dependent effect of these injections in terms of impaired weight gain. While all animals initially lost some weight, control mice and lower doses of antibody had some effect, whereas with the highest dose of antibody, overall weight reduction persisted. There was a dose-dependent reduction in food intake (FIG. 1B), as well as a dose-dependent transcriptional increase for leptin in brown and epididymal adipose tissue, with more limited compensatory effects in subcutaneous adipose tissue (FIG. 1C). Antibody treatment leads to an effective 40% reduction of circulating leptin levels, sufficient to achieve a significant degree of leptin sensitization. Leptin-neutralization not only effectively reduces the weight, but with the weight reduction we also see the expected improvements in glucose homeostasis (FIG. 1D). Furthermore, high fat diet generally affects brown adipose tissue negatively and lead to an increased “whitening” of BAT, and this process is significantly prevented and/or reversed by our antibody treatment (FIG. 1E). Concomitantly, we also observed a reduction in hepatic steatosis.

TABLE 1
CDR* sequences of heavy chain
variable sequences of leptin antibodies

| Name | CDR1 | CDR2 | CDR3 |
|---|---|---|---|
| hLept-1 | Ggtggctccgtcagc | Atccacaccgatggg | Gcgagagagcccggg |
| | agaggtagtcactac | agcacc | ggcgccctgaatttc |
| | (SEQ ID NO: 26) | (SEQ ID NO: 27) | (SEQ ID NO: 28) |
| | GGSVSRGSHY | IHTDGST | AREPGGALNF |
| | (SEQ ID NO: 1) | (SEQ ID NO: 2) | (SEQ ID NO: 3) |
| hLept-2 | Ggatacaccttcacc | Atcaaccctaacagt | Gcgagtgggaaaacg |
| | ggctactat | ggtggcaca | tattacgatttttgg |
| | (SEQ ID NO: 29) | (SEQ ID NO: 30) | agtggtgggagacgc |
| | | | ggtatggacgtc |
| | | | (SEQ ID NO: 31) |
| | GYTFTGYY | INPNSGGT | ASGKTYYDFWSGGRR |
| | (SEQ ID NO: 4) | (SEQ ID NO: 5) | GMDV |
| | | | (SEQ ID NO: 6) |
| hLept-3 | Ggaggcaccttcagc | Atcatccctatcttt | Gcgagaagccaggta |
| | agctatgct | ggtacagca | ccatcctcctactac |
| | (SEQ ID NO: 32) | (SEQ ID NO: 33) | tacggtatggacgtc |
| | | | (SEQ ID NO: 34) |
| | GGTFSSYA | IIPIFGTA | ARSQVPSSYYYGMDV |
| | (SEQ ID NO: 7) | (SEQ ID NO: 8) | (SEQ ID NO: 9) |
| hLept-5 | Ggattcaccttcagt | Atatcatatgatgga | Gcgagaggtcgtgaa |
| | agctatgct | agcaataaa | tactactactacatg |
| | (SEQ ID NO: 35) | (SEQ ID NO: 36) | gacgtc |
| | | | (SEQ ID NO: 37) |
| | GFTFSSYA | ISYDGSNK | ARGREYYYYMDV |
| | (SEQ ID NO: 10) | (SEQ ID NO: 11) | (SEQ ID NO: 12) |
| hLept-6 | Ggatacaccttcacc | Atcaaccctagtggt | Gcgagaggattcggc |
| | agctactat | ggtagcaca | tacggtggtaaggcc |
| | (SEQ ID NO: 38) | (SEQ ID NO: 39) | cttgactac |
| | | | (SEQ ID NO: 40) |
| | GYTFTSYY | INPSGGST | ARGFGYGGKALDY |
| | (SEQ ID NO: 13) | (SEQ ID NO: 14) | (SEQ ID NO: 15) |

*CDRs are defined based on online antibody sequence analytical tool (IMGT).

TABLE 2
List of CDR sequences for
light chain of each antibodies

| Name | CDR1 | CDR2 | CDR3 |
|---|---|---|---|
| hLept-1 | Agctccaacatcgga | agtaa | Gcatcatgggatgac |
| | agtaatact | taat | agcctgaatggtgtg |
| | (SEQ ID NO: 41) | | gta |
| | | | (SEQ ID NO: 42) |
| | SSNIGSNT | SNN | ASWDDSLNGVV |
| | (SEQ ID NO: 16) | | (SEQ ID NO: 17) |
| hLept-2 | Cagagcgttagcagg | acttc | Caacagacttacagt |
| | tat | atcc | accccgtggacg |
| | (SEQ ID NO: 43) | | (SEQ ID NO: 44) |
| | QSVSRY | TSS | QQTYSTPWT |
| | (SEQ ID NO: 18) | | (SEQ ID NO: 19) |
| hLept-3 | Aactccaacatcggg | ggtga | Cagtcctatgacaga |
| | gcaggttatcat | cact | agccggggtggttgg |
| | (SEQ ID NO: 45) | | ttt |
| | | | (SEQ ID NO: 46) |
| | NSNIGAGYH | GDT | QSYDRSRGGWF |
| | (SEQ ID NO: 20) | | (SEQ ID NO: 21) |
| hLept-5 | Aacattgcaagaaaa | aatga | caggtgtgggataat |
| | agt | taac | agtgattatgtc |
| | (SEQ ID NO: 47) | | (SEQ ID NO: 48) |
| | NIARKS | NDN | QVWDNSDYV |
| | (SEQ ID NO: 22) | | (SEQ ID NO: 23) |
| hLept-6 | Cagaatattaatagt | aaggc | Caacagtttgataaa |
| | agg | gtct | tattcgatcact |
| | (SEQ ID NO: 49) | | (SEQ ID NO: 50) |
| | QNINSR | KAS | QQFDKYSIT |
| | (SEQ ID NO: 24) | | (SEQ ID NO: 25) |

TABLE 3
Binding affinities of anti-Leptin antibodies determined by ELISA

| Antibody name | EC50 |
|---|---|
| hLept-1 | 1-500 nM |
| hLept-2 | 1-500 nM |
| hLept-3 | 1-500 nM |
| hLept-5 | 1-500 nM |
| hLept-6 | 1-500 nM |

APPENDIX I
Variable DNA sequences of anti-leptin antibodies

>hLept1_HCv
(SEQ ID NO: 51)
CAGGTACAGCTGCAGCAGTTGGGCCCAGGACTGGTGAAGCCTTCGGAGACCCTGTCCCTCACCT
GCAATGTCTCTGGTGGCTCCGTCAGCAGAGGTAGTCACTACTGGACCTGGATCCGGCAGCCCCC
AGGAAAGGGACTGGAGTGGATTGGGTATATCCACACCGATGGGAGCACCAACTTCAATCCCTCC
CTCAAGAGTCGAGTCACCATGTCACTAGACAGGTCCAGGAACCAGTTCTCCCTGACGCTGAGCT
CTGTGACCGCTACGGACACGGCCGTTTATTATTGTGCGAGAGAGCCCGGGGGCGCCCTGAATTT
CTGGGGCCAGGGAACCCTGGTCACCGTCTCCTCA
>hLept1_LCv
(SEQ ID NO: 52)
AATTTTATGCTGACTCAGCCACCCTCAACGTCTGGGACCCCCGGGCAGAGGGTCACCATCTCTT
GTTCTGGAAGCAGCTCCAACATCGGAAGTAATACTGTAAACTGGTACCAGCAGCTCCCAGGAAC
GGCCCCCAAACTCCTCATCTATAGTAATAATCAGCGGCCCTCAGGGGTCCCTGACCGATTCTCT
GGCTCCAAGTCTGGCACCTCAGCCTCCCTGGCCATCAGTGGGCTCCAGTCTGAGGATGAGGCTG
ATTATTATTGTGCATCATGGGATGACAGCCTGAATGGTGTGGTATTCGGCGGAGGGACCACGGT
GACCGTCCTG
>hLept2_HCv
(SEQ ID NO: 53)
CAGGTGCAGCTGGTGCAGTCTGGGGCTGAGGTGAAGAAGCCTGGGGCCTCAGTGAAGGTCTCCT
GCAAGGCTTCTGGATACACCTTCACCGGCTACTATATGCACTGGGTGCGACAGGCCCCTGGACA
AGGGCTTGAGTGGATGGGACGGATCAACCCTAACAGTGGTGGCACAAACTATGCACAGAAGTTT
CAGGGCAGGGTCACCATGACCAGGGACACGTCCATCAGCACAGCCTACATGGAGCTGAGCAGGC
TGAGATCTGACGACACGGCCGTGTATTACTGTGCGAGTGGGAAAACGTATTACGATTTTTGGAG
TGGTGGGAGACGCGGTATGGACGTCTGGGGCCAAGGGACAATGGTCACCGTCTCTTCA
>hLept2_LCv
(SEQ ID NO: 54)
GACATCCAGTTGACCCAGTCTCCATCCTCCCTGTCTGCATCTGTAGGAGACAGAGTCACCATCA
CTTGCCGGGCAGGTCAGAGCGTTAGCAGGTATTTAAATTGGTTTCAGCAGAAACCAGGGAAAGC
CCCTAAGCTCCTCATCTATACTTCATCCAATTTGCAAAGTGGGGTCCCATCAAGGTTCAGTGCC
AGTGGATCTGGGACAGATTTCACTCTCACCATCAGCAGTCTGCAACCTGAGGATTTTGCTACCT
ACTACTGTCAACAGACTTACAGTACCCCGTGGACGTTCGGCCAAGGGACCAAGCTGGAGATCAA
A
>hLept3_HCv
(SEQ ID NO: 55)
CAGGTGCAGCTGGTGCAGTCTGGGGCTGAGGTGAAGAAGCCTGGGTCCTCGGTGAAGGTCTCCT
GCAAGGCTTCTGGAGGCACCTTCAGCAGCTATGCTATCAGCTGGGTGCGACAGGCCCCTGGACA
AGGGCTTGAGTGGATGGGAGGGATCATCCCTATCTTTGGTACAGCAAACTACGCACAGAAGTTC
CAGGGCAGAGTCACGATTACCGCGGACGAATCCACGAGCACAGCCTACATGGAGCTGAGCAGCC
TGAGATCTGAGGACACGGCCGTGTATTACTGTGCGAGAAGCCAGGTACCATCCTCCTACTACTA
CGGTATGGACGTCTGGGGCCAAGGGACAATGGTCACCGTCTCCTCA
>hLept3_LCv
(SEQ ID NO: 56)
CAGTCTGTGCTGACTCAGCCGCCCTCAGTGTCTGGGGCCCCAGGGCAGAGGGTCACCATCTCCT
GCACTGGGGGCAACTCCAACATCGGGGCAGGTTATCATGTACATTGGTACCAGCAACTTCCAGG
AGCAGCCCCCAAACTCCTCATCTATGGTGACACTAATCGGCCCTCAGGGGTCCCTGACCGATTC
TCTGGCTCTCAGTCTGGCACCTCAGCCTCCCTGGCCATCACTGGGCTCCAGGCTGACGATGAGG
CTGATTATTACTGCCAGTCCTATGACAGAAGCCGGGGTGGTTGGTTTTTCGGCGGAGGGACCCA
GCTGACCGTCCTA
>hLept5_HCv
(SEQ ID NO: 57)
CAGGTGAAGCTGGTGGAGTGGTCGCTGAGCGTGGTCCAGCCTGGGAGGTCCCTGAGACTCTCCT
GCGCAGCCTCTGGATTCACCTTCAGTAGCTATGCTATGCACTGGGTCCGCCAGGCTCCAGGCAA
GGGGCTGGAGTGGGTGGCAGTTATATCATATGATGGAAGCAATAAATACTACGCAGACTCCGTG
AAGGGCCGATTCACCATCTCCAGAGACAATTCCAAGAACACGCTGTATCTGCAAATGAACAGCC
TGAGAGCTGAGGACACGGCTGTGTATTACTGTGCGAGAGGTCGTGAATACTACTACTACATGGA
CGTCTGGGGCAAAGGGACCACGGTCAGCGTCTCCTCA
>hLept5_LCv
(SEQ ID NO: 58)
CAGTCTGTGCTGACTCAGCCACCCTCAGTGTCAGTGGCCCCAGGAAAGACGGCCAGGATTACAT
GTGGGGGATACAACATTGCAAGAAAAAGTGTGCACTGGTACCAGCAGAAGCCAGGCCAGGCCCC
TGTGTTGGTCATGTATAATGATAACGACCGGCCCTCAGGGATCCCTGAGCGATTCTCTGGCTCC
AACTCTGGGAACACGGCCACCCTGACCATCAGCAGGGTCGAAGCCGGGGATGAGGCCGACTATT
ACTGTCAGGTGTGGGATAATAGTGATTATGTCTTCGGAACTGGGACCAAGGTCACCGTCCTA
>hLept6_HCv
(SEQ ID NO: 59)
CAGGTGCAGTTGATGCAGTCTGGGGCTGAGGTGAAGAAGCCTGGGGCCTCAGTGAAGGTTTCCT
GCAAGGCATCTGGATACACCTTCACCAGCTACTATATGCACTGGGTGCGACAGGCCCCTGGACA
AGGGCTTGAGTGGATGGGAATAATCAACCCTAGTGGTGGTAGCACAAGCTACGCACAGAAGTTC
CAGGGCAGAGTCACCATGACCAGGGACACGTCCACGAGCACAGTCTACATGGAGCTGAGCAGCC
TGAGATCTGAGGACACGGCCGTGTATTACTGTGCGAGAGGATTCGGCTACGGTGGTAAGGCCCT
TGACTACTGGGGCCAGGGAACCACGGTCACCGTCTCTTCA
>hLept6_LCv
(SEQ ID NO: 60)
GACATCCAGATGACCCAGTCTCCTCCCACCCTGTCTGCATCTGTAGGGGACAGAGTCACCATCA
CTTGCCGGGCCAGTCAGAATATTAATAGTAGGTTGGCCTGGTATCAGCAGAAACCAGGGAGAGC
CCCTAAACTCCTGATCTATAAGGCGTCTACTTTAGAGAGTGGGGTCCCATCGAGGTTCAGCGGC
AGTGGATCTGGGACAGAATTCACTCTCACCATCAGCAGCCTGCAGCCAGATGACTTTGCAACTT
ATTACTGCCAACAGTTTGATAAATATTCGATCACTTTCGGCGGAGGGACCAAGATGGAGATCAA
A

APPENDIX II
Variable amino acid sequences
of anti-leptin antibodies

>hLept1_HCv_AA
(SEQ ID NO: 61)
QVQLQQLGPGLVKPSETLSLTCNVSGGSVSRGSHYWTWIRQPPGKGLEWI
GYIHTDGSTNFNPSLKSRVTMSLDRSRNQFSLTLSSVTATDTAVYYCARE
PGGALNFWGQGTLVTVSS
>hLept1_LCv_AA
(SEQ ID NO: 62)
DFMLTQPPSTSGTPGQRVTISCSGSSSNIGSNTVNWYQQLPGTAPKLLIY
SNNQRPSGVPDRFSGSKSGTSASLAISGLQSEDEADYYCASWDDSLNGVV
FGGGTTVTVL
>hLept2_HCv_AA
(SEQ ID NO: 63)
QVQLVQSGAEVKKPGASVKVSCKASGYTFTGYYMHWVRQAPGQGLEWMGR
INPNSGGTNYAQKFQGRVTMTRDTSISTAYMELSRLRSDDTAVYYCASGK
TYYDFWSGGRRGMDVWGQGTMVTVSS
>hLept2_LCv_AA
(SEQ ID NO: 64)
DIQLTQSPSSLSASVGDRVTITCRAGQSVSRYLNWFQQKPGKAPKLLIYT
SSNLQSGVPSRFSASGSGTDFTLTISSLQPEDFATYYCQQTYSTPWTFGQ
GTKLEIK
>hLept3_HCv_AA
(SEQ ID NO: 65)
QVQLVQSGAEVKKPGSSVKVSCKASGGTFSSYAISWVRQAPGQGLEWMGG
IIPIFGTANYAQKFQGRVTITADESTSTAYMELSSLRSEDTAVYYCARSQ
VPSSYYYGMDVWGQGTMVTVSS
>hLept3_HCv_AA
(SEQ ID NO: 66)
QSVLTQPPSVSGAPGQRVTISCTGGNSNIGAGYHVHWYQQLPGAAPKLLI
YGDTNRPSGVPDRFSGSQSGTSASLAITGLQADDEADYYCQSYDRSRGGW
FFGGGTQLTVL
>hLept5_HCv_AA
(SEQ ID NO: 67)
QVKLVEWSLSVVQPGRSLRLSCAASGFTFSSYAMHWVRQAPGKGLEWVAV
ISYDGSNKYYADSVKGRFTISRDNSKNTLYLQMNSLRAEDTAVYYCARGR
EYYYYMDVWGKGTTVSVSS
>hLept5_LCv_AA
(SEQ ID NO: 68)
QSVLTQPPSVSVAPGKTARITCGGYNIARKSVHWYQQKPGQAPVLVMYND
NDRPSGIPERFSGSNSGNTATLTISRVEAGDEADYYCQVWDNSDYVFGTG
TKVTVL
>hLept6_HCv_AA
(SEQ ID NO: 69)
QVQLMQSGAEVKKPGASVKVSCKASGYTFTSYYMHWVRQAPGQGLEWMGI
INPSGGSTSYAQKFQGRVTMTRDTSTSTVYMELSSLRSEDTAVYYCARGF
GYGGKALDYWGQGTTVTVSS
>hLept6_LCv_AA
(SEQ ID NO: 70)
DIQMTQSPPTLSASVGDRVTITCRASQNINSRLAWYQQKPGRAPKLLIYK
ASTLESGVPSRFSGSGSGTEFTLTISSLQPDDFATYYCQQFDKYSITFGG
GTKMEIK

1-4. (canceled)

5. An antibody or an antigen binding fragment thereof, wherein the antibody or antigen binding fragment thereof comprises HCDR1, HCDR2, and HCDR3 amino acid sequences provided in a heavy chain variable domain (VH) amino acid sequence and LCDR1, LCDR2, and LCDR3 amino acid sequences provided in a light chain variable domain (VL) amino acid sequence wherein the VH and VL amino acid sequences are selected from the group consisting of 63 and 64, 67 and 68, and 69 and 70.

6. The antibody or antigen binding fragment thereof of claim 5, wherein the HCDR1, HCDR2, and HCDR3 comprise the amino acid sequences of SEQ ID NOs: 4, 5, and 6, respectively.

7. The antibody or antigen binding fragment thereof of claim 5, wherein the LCDR1, LCDR2, and LCDR3 comprise the amino acid sequences of SEQ ID NO: 18, TSS, and SEQ ID NO: 19, respectively.

8. The antibody or antigen binding fragment thereof of claim 5, wherein the antibody or an antigen binding fragment thereof comprises a VH comprising an amino acid sequence at least 95% identical to the amino acid sequence of SEQ ID NO: 63.

9. The antibody or antigen binding fragment thereof of claim 5, wherein the antibody or an antigen binding fragment thereof comprises a VL comprising an amino acid sequence at least 95% identical to the amino acid sequence of SEQ ID NO: 64.

10. The antibody or antigen binding fragment thereof of claim 8, wherein the VH comprises the amino acid sequence of SEQ ID NO: 63.

11. The antibody or antigen binding fragment thereof of claim 9, wherein the VL comprises the amino acid sequence of SEQ ID NO: 64.

12. The antibody or antigen binding fragment thereof of claim 5, wherein the HCDR1, HCDR2, and HCDR3 comprise the amino acid sequences of SEQ ID NOs: 10, 11, and 12, respectively.

13. The antibody or antigen binding fragment thereof of claim 5, wherein the LCDR1, LCDR2, and LCDR3 comprise the amino acid sequences of SEQ ID NO: 22, NDN, and SEQ ID NO: 23, respectively.

14. The antibody or antigen binding fragment thereof of claim 5, wherein the antibody or an antigen binding fragment thereof comprises a VH comprising an amino acid sequence at least 95% identical to the amino acid sequence of SEQ ID NO: 67.

15. The antibody or antigen binding fragment thereof of claim 5, wherein the antibody or an antigen binding fragment thereof comprises a VL comprising an amino acid sequence at least 95% identical to the amino acid sequence of SEQ ID NO: 68.

16. The antibody or antigen binding fragment thereof of claim 14, wherein the VH comprises the amino acid sequence of SEQ ID NO: 67.

17. The antibody or antigen binding fragment thereof of claim 15, wherein the VL comprises the amino acid sequence of SEQ ID NO: 68.

18. The antibody or antigen binding fragment thereof of claim 5, wherein the HCDR1, HCDR2, and HCDR3 comprise the amino acid sequences of SEQ ID NOs: 13, 14, and 15, respectively.

19. The antibody or antigen binding fragment thereof of claim 5, wherein the LCDR1, LCDR2, and LCDR3 comprise the amino acid sequences of SEQ ID NO: 24, KAS, and SEQ ID NO: 25, respectively.

20. The antibody or antigen binding fragment thereof of claim 5, wherein the antibody or an antigen binding fragment thereof comprises a VH comprising an amino acid sequence at least 95% identical to the amino acid sequence of SEQ ID NO: 69.

21. The antibody or antigen binding fragment thereof of claim 5, wherein the antibody or an antigen binding fragment thereof comprises a VL comprising an amino acid sequence at least 95% identical to the amino acid sequence of SEQ ID NO: 70.

22. The antibody or antigen binding fragment thereof of claim 20, wherein the VH comprises the amino acid sequence of SEQ ID NO: 69.

23. The antibody or antigen binding fragment thereof of claim 21, wherein the VL comprises the amino acid sequence of SEQ ID NO: 70.
